(19) United States
(12) Patent Application Publication
(10) Pub. No.: US 2022/0145031 A1
(43) Pub. Date: May 12, 2022

(54) COMPOSITE ARTICLE INCLUDING AMORPHOUS CARBON COATING

(71) Applicant: SAINT-GOBAIN PERFORMANCE PLASTICS CORPORATION, Solon, OH (US)

(72) Inventors: Xipeng LIU, Concord, MA (US); Jian L. DING, Stow, MA (US); Adam P. NADEAU, Boyston, MA (US); Yahya Motemani SHARABIANI, Aachen (DE); Jan HAGEN, Bonn (DE); Gayla LYON, West Boylston, MA (US)

(21) Appl. No.: 17/454,593

(22) Filed: Nov. 11, 2021

Related U.S. Application Data
(60) Provisional application No. 63/112,988, filed on Nov. 12, 2020.

(51) Int. Cl. C08J 7/06; A61L 29/06; A61L 29/10; C08L 83/04

(52) U.S. Cl. CPC C08J 7/06; A61L 29/06; C08K 3/36; C08L 83/04; A61L 29/103

(57) ABSTRACT

A composite article including a substrate, having a first major surface, a second major surface; and an amorphous carbon coating overlying the first or the second major surface, where the substrate includes a composition of 1-99 wt. % of an organic polymer and 1-99 wt. % of an inorganic filler.

### CROSS-REFERENCE TO RELATED APPLICATION(S)

This application claims priority under 35 U.S.C. § 119(e) to U.S. Provisional Application No. 63/112,988, entitled “COMPOSITE ARTICLE INCLUDING AMORPHOUS CARBON COATING,” by Xipeng LIU et al., filed Nov. 12, 2020, which is assigned to the current assignee hereof and incorporated herein by reference in its entirety.

### FIELD OF THE DISCLOSURE

This disclosure, in general, relates to composite articles with an amorphous carbon coating.

### BACKGROUND

Composite materials including a composition of substrates and fillers are generally known. Often, these composite materials may be used in pharmaceutical manufacturing, medical device, food and beverage, fluid transfer, aerospace, vehicle, and habitat applications. Often in these applications, composite materials with higher performance in chemical inertness, biocompatibility, elasticity, low coefficient of friction, resistance to dust contamination, low vapor permeability, low surface electrical resistivity and temperature resistance are desired. As a result, there is an ongoing need for improved composite articles having optimal values in chemical inertness, biocompatibility, elasticity, low coefficient of friction, resistance to dust contamination, low vapor permeability, low surface electrical resistivity and temperature resistance for their desired application.

### BRIEF DESCRIPTION OF THE DRAWINGS

The present disclosure may be better understood, and its numerous features and advantages made apparent to those skilled in the art by referencing the accompanying drawings.

FIG. 1A shows a cross section illustrating the various layers of the composite article.

FIG. 1B shows a cross section illustrating the various layers of the composite article.

FIG. 2A shows an illustration of an exemplary medical device incorporating the composite article.

FIG. 2B shows an illustration of an exemplary medical device incorporating the composite article.

FIG. 3A shows an illustration of an exemplary medical device incorporating the composite article.

FIG. 3B shows an illustration of an exemplary medical device incorporating the composite article.

FIG. 4A shows an illustration of a Raman spectroscopy incorporating the composite article.

FIG. 4B shows an illustration of a Raman spectroscopy incorporating the composite article.

FIG. 4C shows an illustration of a Raman spectroscopy incorporating the composite article.

FIG. 5 shows an illustration of a surface resistivity of composite articles according to embodiments herein and composite articles known in the art.

The use of the same reference symbols in different drawings indicates similar or identical items.

### DETAILED DESCRIPTION

The following description in combination with the figures is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other embodiments can be used based on the teachings as disclosed in this application.

As used herein, the phrase “engagement element” refers to a feature disposed on an outer surface or on an inner diameter of the port, such as in bore of the port, that is adapted to engage with a separate element from the port. An engagement element can include protrusions, such as a barb, depressions, recesses, or any other structure that is adapted to engage with a separate element from the port when making a sterile connection.

As used herein, a “vessel” refers to any structure capable of receiving and a holding a fluid, which could include liquids, gases, or combinations thereof. A vessel includes, but is not limited to an open vessel, a closed vessel, a rigid vessel, a flexible vessel, a transparent vessel, a bag such as a mixing bag or an isolation bag, a drum, a container, a room, or any other structure to which a sterile connection is desired.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item may be used in place of a single item. Similarly, where more than one item is described herein, a single item may be substituted for that more than one item.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the composite material arts.

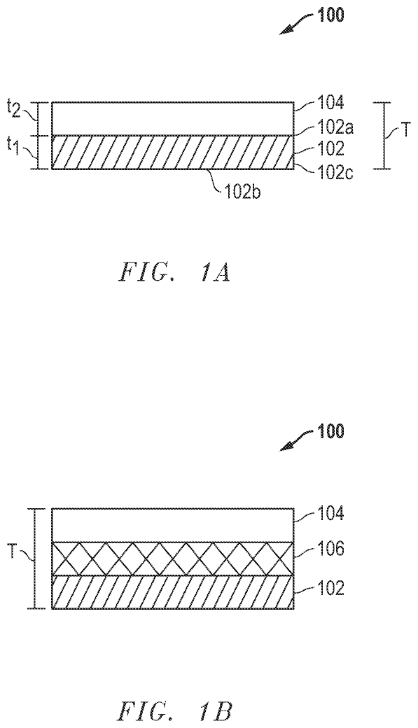

FIG. 1A shows a cross section illustrating the various layers of the composite article, generally designated 100. Composite article 100 can include a substrate 102. The substrate 102 can have a first major surface 102a and a second major surface 102b separated by an edge 102c located between the first major surface 102a and the second major surface 102b. The substrate 102 may include a composition of an organic polymer and an inorganic filler. The substrate 102 can include an organic polymer selected from an polydimethylsiloxane (PDMS), functionally modified polydimethylsiloxane, polytetrafluoroethylene (PTFE), fluorinated ethylene-propylene (FEP), polyvinylidenfluoride (PVDF), polychlorotrifluoroethylene (PCTFE), ethylene chlorotrifluoroethylene (ECTFE), perfluoroalkoxypolymer (PFA), polyacetal (POM), polybutylene terephthalate (PBT), polyimide (PI), polyamidimide (PAI), polyetherimide, polyetheretherketone (PEEK), polyethylene, such as ultrahigh molecular weight polyethylene (UHMWPE), polysulfone, polyamide, polyphenylene oxide, polyphenylene sulfide (PPS), polyurethane, polyester, ethylene-tetrafluoroethylene (ETFE), tetrafluoro-ethylene-perfluoro (methyl vinyl ether) (MFA), polyvinylidene fluoride (PVDF), ethylene-chlorotrifluoroethylene (ECTFE), polyimide (PI), polyamidimide (PAI), polyphenylene sulfide (PPS), polyethersulofone (PES), polyphenylene sulfone (PPSO2), liquid crystal polymers (LCP), polyetherketone (PEK), polyether ether ketones (PEEK), aromatic polyesters (Ekonol), polyetherketone (PEK), liquid crystal polymer (LCP), polyamide (PA), polyethylene (PE), polypropylene (PP), polystyrene, styrene butadiene copolymers, polyesters, polycarbonate, polyacrylonitriles, polyamides, styrenic block copolymers, ethylene vinyl alcohol copolymers, ethylene vinyl acetate copolymers, polyesters grafted with maleic anhydride, poly-vinylidene chloride, aliphatic polyketone, liquid crystalline polymers, ethylene methyl acrylate copolymer, ethylene-norbornene copolymers, polymethylpentene and ethylene acrylic acid copoloymer, mixtures, copolymers, or any combination thereof. In a particular embodiment, the substrate 102 can include an elastomer selected from nitrile rubber, neoprene rubber, silicone rubber, an olefinic elastomer, a styrene elastomer, a thermoplastic elastomer, a crosslinked elastomer, a polyether-polyester elastomer, an ethylene-propylene elastomer, ethylene-acrylate rubbers, a fluoro elastomer, or any combination thereof. In one further embodiment, the substrate 102 and/or silicone elastomer can include vinyl-containing polydimethylsiloxane. In one further embodiment, the substrate 102 and/or silicone elastomer can include a hydride-containing polysiloxane cross-linker. In one further embodiment, the substrate 102 and/or silicone elastomer can include a metal catalyst selected from the group of platinum, boron, lithium, beryllium, sodium, magnesium, scandium, titanium, vanadium, chromium, manganese, iron, cobalt, nickel, copper, silicon, tin, aluminum, or zinc. The substrate 102 can include an inorganic filler selected from glass, glass fibers, carbon, carbon fibers, silicon, graphite, PEEK, molybdenum disulfide, aromatic polyester, carbon particles, bronze, fluoropolymer, thermoplastic fillers, silicon carbide, aluminum oxide, silicon dioxide, polyamidimide (PAI), polyimide (PI), PPS, polyphenylene sulfone (PPSO2), liquid crystal polymers (LCP), aromatic polyesters (Econol), and mineral particles such as wollastonite, CaF₂, barium sulfate, or any combination thereof. The inorganic filler can include silica.

The composition of the substrate 102 can include a composition of an organic polymer and an inorganic filler. In a number of embodiments, the substrate 102 may have a content of organic polymer of at least 20 wt. %, at least 30 wt. %, at least 40 wt. %, at least 50 wt. %, at least 60 wt. %, at least 70 wt. %, at least 75 wt. %, at least 80 wt. %, at least 85 wt. %, or at least 90 wt. %, or at least 95 wt. %, or at least 99 wt. %. In a number of embodiments the substrate 102 may have a content of organic polymer of no greater than 99 wt. %, no greater than 95 wt. %, no greater than 90 wt. %, no greater than 80 wt. %, no greater than 75 wt. %, no greater than 60 wt. %, no greater than 50 wt. %, no greater than 35 wt. %, no greater than 25 wt. %, no greater than 15 wt. %, no greater than 10 wt. %, no greater than 5 wt. %, or no greater than 1 wt. %. In a number of embodiments, the substrate 102 may have a content of inorganic filler of at least 1 wt. %, at least 5 wt. %, at least 20 wt. %, at least 30 wt. %, at least 40 wt. %, at least 50 wt. %, at least 60 wt. %, at least 70 wt. %, at least 75 wt. %, at least 80 wt. %, at least 85 wt. %, or at least 90 wt. %, at least 95 wt. %, or at least 99%. In a number of embodiments the substrate 102 may have a content of inorganic filler of no greater than 99 wt. %, no greater than 95 wt. %, no greater than 90 wt. %, no greater than 80 wt. %, no greater than 75 wt. %, no greater than 60 wt. %, no greater than 50 wt. %, no greater than 35 wt. %, no greater than 25 wt. %, no greater than 15 wt. %, no greater than 10 wt. %, no greater than 5 wt. %, or no greater than 1%.

The substrate 102 has a thickness t₁. In an embodiment, t₁ is at least 0.025 mm, at least 0.050 mm, at least 0.100 mm, at least 0.500 mm, at least 1 mm, at least 2 mm, at least 3 mm, at least 4 mm, at least 5 mm, at least 6 mm, at least 8 mm, at least 10 mm, at least 50 mm, at least 80 mm, at least 100 mm, at least 150 mm, at least 200 mm, or at least 300 mm. In another embodiment, t₁ is not greater than 800 mm, not greater than 700 mm, not greater than 650 mm, not greater than 600 mm, not greater than 550 mm, not greater than 500 mm, not greater than 480 mm, not greater than 460 mm, not greater than 440 mm, or not greater than 420 mm. In yet one further embodiment, t₁ ranges from 50 mm to 800 mm, such as from 100 mm to 600 mm, from 200 mm to 500 mm, or from 300 mm to 450 mm. In one particular embodiment, t₁ ranges from 380 mm to 420 mm.

Referring back to FIG. 1A, the composite article 100 can include an amorphous carbon coating 104. The amorphous carbon coating 104 can be coated such that it overlies at least one of the first 102a, second 102b major surface of the substrate 102. The amorphous carbon coating 104 can be coated such that it overlies an edge 102c of the substrate 102. The amorphous carbon coating may include a composition of diamond-like carbon content and graphite carbon content. In other words, the amorphous carbon coating 104 may be characterized by the ratio between SP3 and SP2 carbon-carbon bonds The composite article 100 may include an amorphous carbon coating 104 having an SP3 diamond-like carbon content of at least 20 wt. %, at least 30 wt. %, at least 40 wt. %, at least 50 wt. %, at least 60 wt. %, at least 70 wt. %, at least 75 wt. %, at least 80 wt. %, at least 85 wt. %, at least 90 wt. %, at least 95 wt. %, or at least 99 wt. %. The composite article 100 may include an amorphous carbon coating 104 having an SP2 graphite carbon content of at least 2%, at least 5%, at least 10%, at least 20%, at least 30%, at least 40%, at least 50%, at least 60%, at least 70%, at least 80%, at least 85 wt. %, at least 90 wt. %, at least 95 wt. %, or at least 99 wt. %.

The amorphous carbon coating 104 has a thickness t₂. In one embodiment, t₂ can be at least 10 Å, at least 20 Å, at least 30 Å, at least 50 Å, at least 80 Å, at least 100 Å, at least 150 Å, at least 200 Å, at least 300 Å, at least 400 Å, at least 500 Å, at least 600 Å, at least 800 Å, at least 1000 Å, at least 1200 Å, at least 1400 Å, at least 1600 Å, at least 1800 Å, least 2000 Å, or even at least 5000 Å. In another embodiment, t₂ is not greater than 5500 Å, not greater than 2500 Å, not greater than 2000 Å, not greater than 1800 Å, not greater than 1600 Å, not greater than 1400 Å, not greater than 1200 Å, not greater than 1000 Å, not greater than 800 Å, not greater than 700 Å, not greater than 650 Å, not greater than 600 Å, not greater than 550 Å, not greater than 500 Å, not greater than 480 Å, not greater than 460 Å, not greater than 440 Å, or not greater than 420 Å. In yet one further embodiment, t₂ is in the range from 10 Å to 2000 Å, in a range from 50 Å to 1000 Å, in a range from 100 Å to 500 Å, or in a range from 100 Å to 200 Å.

FIG. 1B shows a cross section illustrating the various layers of a different composite article. In a number of embodiments, an amorphous carbon coating 104 can be applied to the substrate 102 with an adhesion promotion layer 106. The adhesion promotion layer 106 can include a polymer, such as a fluoropolymer. The adhesion promoter layer 106 can include a thermoplastic. Examples of polymers that can be used in adhesion promoter layer 106 include tetrafluoroethylene-hexafluoropropylene (FEP), perfluoroalkoxyethylene (PFA), polyimide (PI), polyamidimide (PAI), polyphenylene sulfide (PPS), polyphenylene sulfone (PPSO2), polyether ether ketones (PEEK), aromatic polyesters (Ekonol), ethylene-tetrafluoroethylene (ETFE), tetrafluoro-ethylene-perfluoro (methyl vinyl ether) (MFA), modified polytetrafluoroethylene (TFM), polyvinylidene fluoride (PVDF), ethylene-chlorotrifluoroethylene (ECTFE), polyethersulfone (PES), polyetherketone (PEK), polyethylene (PE), UHMWPE, or any combination thereof.

The adhesion promotion layer 106 can include a metal element containing compound such as aluminum oxide, silicon oxide, or silanes, siloxane titanium oxide, tantalume oxide, silicone nitride, Titanium and Tantalum.

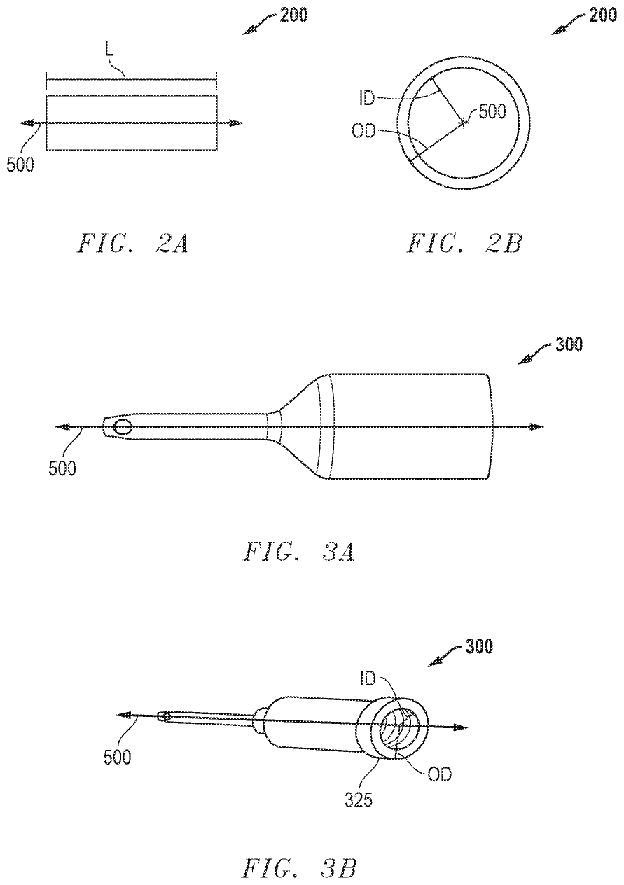

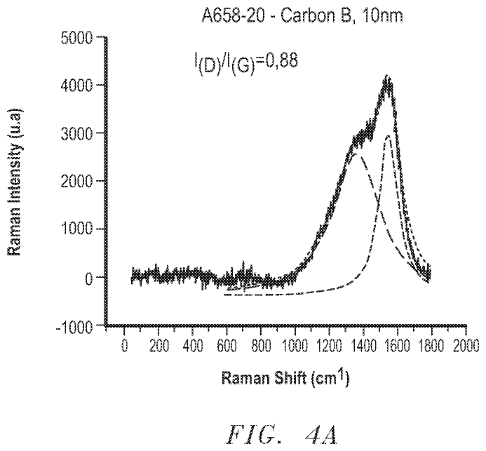

In a number of embodiments, the composite article can be formed into a medical device 200. FIGS. 2A-3B each show a medical device 200 incorporating the composite article according to embodiments herein. FIGS. 2A-2B illustrate a medical device 200 that can include structures having a lumen through which a fluid may be carried, pumped, or otherwise transported through, such as medical sterile tubing. FIGS. 3A and 3B illustrate a medical device 300 that can include an infusion sleeve. The medical device 200, 300 can have substantial transparency (60-80% light transmission in the visible range (TL)). The TL value can be tuned from 5-80%. The composite article 100 or medical device may include an amorphous carbon coating 104 with a refractive index between 1.2 and 2.5.

The composite article or medical device may include an amorphous carbon coating 104 with a density between 2.0 and 3.3 g/cm³.

The medical device 200, 300 can be sterile.

The composite article or medical device can have a Shore A hardness in the range from 5 to 95, in a range from 10 to 80, in a range from 20 to 70, in a range of 30-60, or in a range from 40 to 60.

The composite article or medical device can have a coefficient of friction of 0.01 to 1.6 when tested on stainless steel.

The composite article or medical device can have a surface electrical resistivity (Ω/sq) between 10² and 10¹⁸.

The composite article or medical device can have a high temperature resistance between 150° C. and 260° C. High temperature resistance may be defined as the heat deflection temperature under ASTM D648 with an applied pressure of about 0.455 MPa.

The composite article or medical device can have a low temperature resistance between −110° C. and −30° C.

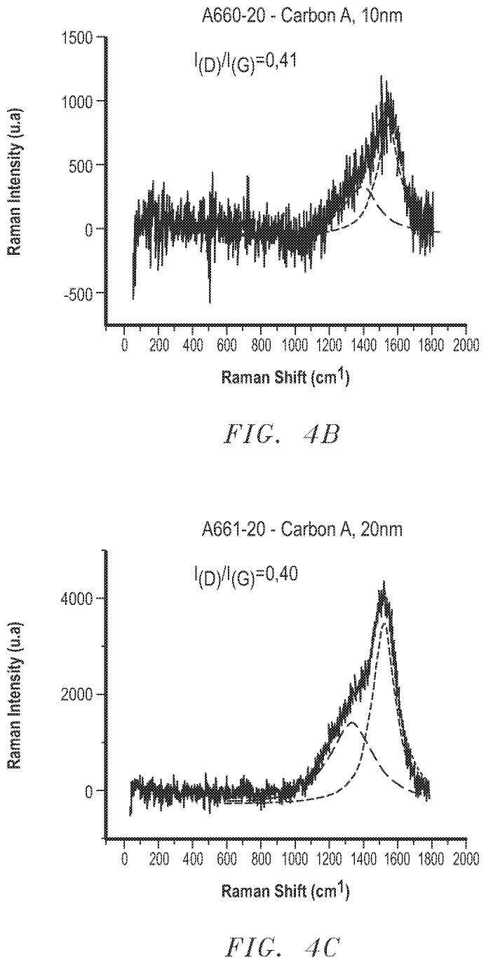

FIGS. 4A-4C show an illustration of a Raman spectroscopy incorporating the composite article or medical device. FIG. 4D illustrates a Raman spectroscopy incorporating the composite article or medical device with a coating of higher SP2 graphite with a coating thickness of about 10 nm. FIG. 4B illustrates a Raman spectroscopy incorporating the composite article 100 with an amorphous carbon coating of higher SP3 diamond-like carbon with a coating thickness of about 10 nm. FIG. 4C illustrates a Raman spectroscopy incorporating the composite article or medical device with an amorphous carbon coating of higher SP3 diamond-like carbon with a coating thickness of about 20 nm. As shown in FIGS. 4A-4C, the composite article or medical device may include an amorphous carbon coating 104 with the following Raman spectroscopy features: selected laser wavelengths of ˜514-532 nm (532 nm); G-band peak position ranging from 1600 cm⁻¹ to 1505 cm⁻¹; and a I (D)/I (G) ranging from 2.00 to 0.05. As used herein, “I” refers to the peak intensity.

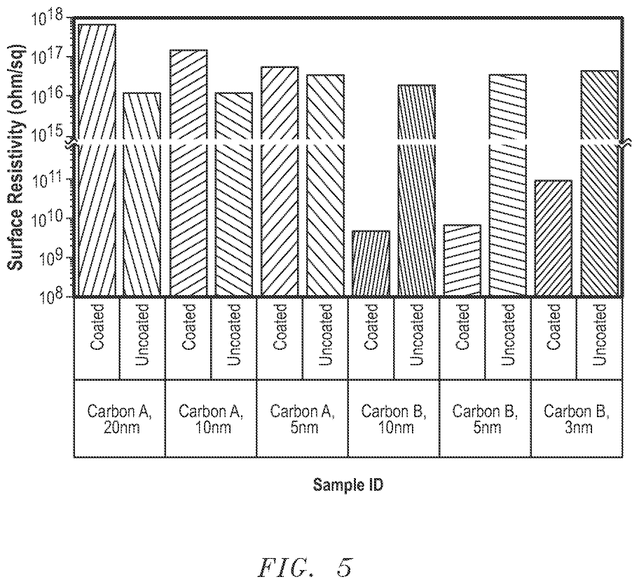

The following Table 1 and FIG. 5 show an illustration of a surface resistivity of composite articles according to embodiments herein and composite articles known in the art. The coated surfaces each show an improved surface resistivity versus the uncoated surfaces at similar thicknesses, temperatures, and applied voltages, as shown in Table 1 and FIG. 5.

TABLE 1

| | | Thick- | Applied | Customer | | Surface |
|---|---|---|---|---|---|---|
| Temperature | % | ness | Voltage | Sample | | Resistivity |
| (° C.) | RH | (cm) | (V) | Reference | Surface | (ohm/sq) |
| 19 | 50 | 0.2000 | 500 | A661-20 - | Coated | 6.40E+17 |
| | | | | Carbon A, | | |
| | | | | 20 nm | | |
| | | | | A661-20 - | Uncoated | 1.20E+16 |
| | | | | Carbon A, | | |
| | | | | 20 nm | | |
| | | | | A660-20 - | Coated | 1.40E+17 |
| | | | | Carbon A, | | |
| | | | | 10 nm | | |
| | | | | A660-20 - | Uncoated | 1.20E+16 |
| | | | | Carbon A, | | |
| | | | | 10 nm | | |
| | | | | A659-20 - | Coated | 5.40E+16 |
| | | | | Carbon A, | | |
| | | | | 5 nm | | |
| | | | | A659-20 - | Uncoated | 3.40E+16 |
| | | | | Carbon A, | | |
| | | | | 5 nm | | |
| | | | | A658-20 - | Coated | 4.60E+09 |
| | | | | Carbon B, | | |
| | | | | 10 nm | | |
| | | | | A658-20 - | Uncoated | 1.80E+16 |
| | | | | Carbon B, | | |
| | | | | 10 nm | | |
| | | | | A657-20 - | Coated | 6.70E+09 |
| | | | | Carbon B, | | |
| | | | | 5 nm | | |
| | | | | A657-20 - | Uncoated | 3.40E+16 |
| | | | | Carbon B, | | |
| | | | | 5 nm | | |
| | | | | A656-20 - | Coated | 9.00E+10 |
| | | | | Carbon B, | | |
| | | | | 3 nm | | |
| | | | | A656-20 - | Uncoated | 4.30E+16 |
| | | | | Carbon B, | | |
| | | | | 3 nm | | |

Customer sample reference Carbon A 20 nm indicates a composite article and or medical device with an amorphous carbon coating of higher diamond-like carbon SP3 content at 20 nm thickness. Customer sample reference Carbon A 10 nm indicates a composite article and or medical device with an amorphous carbon coating of higher diamond-like carbon SP3 content at 10 nm thickness. Customer sample reference Carbon A 5 nm indicates a composite article and or medical device with an amorphous carbon coating of higher diamond-like carbon SP3 content at 5 nm thickness. Customer sample reference Carbon B 5 nm indicates a composite article and or medical device with an amorphous carbon coating of higher graphite carbon SP2 content at 5 nm thickness. Customer sample reference Carbon B 3 nm indicates a composite article and or medical device an amorphous carbon coating of higher graphite carbon SP2 content at 3 nm thickness. Customer sample reference Carbon B 10 nm indicates a composite article and or medical device with an amorphous carbon coating of higher graphite carbon SP2 content at 10 nm thickness. As shown in Table 1, fixed test electrodes were used to test the surface resistivity at a temperate of about 19° C. with a RH % of 50 and applied voltage of 500 V. The surface substrate shown in table 1 was silicone polymer. As shown in Table 1, the amorphous carbon coating of higher graphite carbon SP2 content on the label side of the sample generated lower surface resistivity versus the uncoated side of the sample. Further as shown in Table 1 and FIG. 5, the amorphous carbon coating of higher diamond-like carbon SP3 content on the label side of the sample generated higher surface resistivity versus the uncoated side of the sample.

In a particular embodiment, a silicone sample with coating thickness between 20-200 nm according to embodiments herein showed improved oxygen barrier properties, where the oxygen transmission rate was decreased by 10-100× from the uncoated control samples described above in Table 1.

The composite article 100 and/or medical device 200, 300 can be formed to include any cross-sectional shape. In a number of embodiments, the composite article 100 and/or medical device 200, 300 can have a polygonal cross-section down a central axis 500 (shown best in FIGS. 2A-2B). In a number of embodiments, the composite article 100 and/or medical device 200, 300 can have a polygonal cross-section down a central axis that is regular or non-regular. In a number of embodiments, the composite article 100 and/or medical device 200, 300 can have circular or oval cross-section down a central axis. In a number of embodiments, the composite article 100 and/or medical device 200, 300 can be formed to include at least one axial collar 325. In a number of embodiments, the composite article 100 and/or medical device 200, 300 can be formed to have an essentially cylindrical shape. In a number of embodiments, the composite article 100 and/or medical device 200, 300 can be formed to have a conical shape.

The composite article 100 and/or medical device 200, 300 can have an inner diameter (or smallest dimension), ID, where the inner diameter is at least 0.1 mm, at least 1 mm, at least 2 mm, at least 5 mm, at least 10 mm, at least 25 mm, at least 50 mm, at least 100 mm, at least 200 mm, at least 250 mm, or at least 500 mm. The composite article 100 and/or medical device 200, 300 can have an inner diameter (or smallest dimension), ID, where the inner diameter is not greater than 1000 mm, not greater than 500 mm, not greater than 250 mm, not greater than 200 mm, or not greater than 1500 mm. The composite article 100 and/or medical device 200, 300 can have an inner diameter (or smallest dimension), ID, where the inner diameter is in a range from 0.1 mm to 250 mm, in a range from 1 mm to 200 mm, in a range from 1 mm to 150 mm, or in a range from 1 mm to 125 mm. The composite article 100 and/or medical device 200, 300 can have an inner diameter (or smallest dimension), ID, that varies along a length of the composite article 100 and/or medical device 200, 300.

The composite article 100 and/or medical device 200, 300 can have an outer diameter (or largest dimension), OD, where the outer diameter is at least 0.5 mm, at least 1 mm, at least 2 mm, at least 5 mm, at least 10 mm, at least 25 mm, at least 50 mm, at least 100 mm, at least 200 mm, at least 250 mm, or at least 500 mm. The composite article 100 and/or medical device 200, 300 can have an outer diameter (or largest dimension), OD, where the outer diameter is not greater than 1000 mm, not greater than 500 mm, not greater than 250 mm, not greater than 200 mm, or not greater than 1500 mm. The composite article 100 and/or medical device 200, 300 can have an outer diameter (or largest dimension), OD, where the outer diameter is in a range from 1 mm to 250 mm, in a range from 1 mm to 200 mm, in a range from 5 mm to 150 mm, or in a range from 5 mm to 125 mm. The composite article 100 and/or medical device 200, 300 can have an outer diameter (or largest dimension), OD, that varies along a length of the composite article 100 and/or medical device 200, 300.

Turning to the method of forming the composite article, the amorphous carbon coating can be applied to the substrate using any technique known in the industry. In a number of embodiments, application of the amorphous carbon coating to the substrate may include a surface treatment including physical vapor deposition, chemical vapor disposition, cathodic vacuum arc evaporation, magnetron sputtering, plasma enhanced chemical vapor disposition, spraying, microwave radiation, ultraviolet radiation, x-ray radiation, gamma radiation, alpha radiation, beta radiation, charged ions, neutron radiation, vacuum plasma radiation, cold plasma radiation, other chemical or electrochemical techniques, microwave, electron cyclotron resonance (ECR), microwave integrated with ECR, direct current (DC), RF-glow or any combination thereof. In a number of embodiments, the mixture of gases, frequency signal of the plasma, power output, or another component may determine the diamond-like carbon SP3 and graphite carbon SP2 mix in the amorphous carbon coating.

In an exemplary process to form the composite article according to embodiments herein, amorphous carbon reformed gas is introduced at a controlled rate into a vacuum chamber in which the surface to be modified is located or is passed through the substrate surface. The amorphous carbon modifying gas is any gas or mixture of gases that reacts at the substrate surface to form a carbon deposition or leave a diamond-like coating on the substrate surface. A high frequency signal is applied via an external power source to form plasma. It will be apparent to those skilled in the art that an appropriate frequency can be selected depending on the particular amorphous carbon reformed gas used. In general, frequencies on the order of 10 kHz to 10 MHz are useful in the present invention, although lower or higher frequencies may be used depending on the material used to modify the surface. The output of the RF signal is not limited as long as it is sufficient to ignite the plasma and promote coating. A power density of 0.01 kW/cm² to 10 kW/cm² can be used. The plasma is ignited in the chamber and maintained for a selected time at a preselected power setting. When processing is complete, the radio frequency switch is turned off to extinguish the plasma. The chamber is then flushed and the product is collected. As a result of the treatment, a thin layer of amorphous carbon is deposited on the modified surface. The thickness of the layer is about 10 to 10,000 Å, for example 50 to 5000 Å. In some embodiments of the present invention, the surface modification of amorphous carbon can be repeated one or more times. A series of successive deposition steps help provide an amorphous carbon coating that is well adhered to the substrate surface and is uniformly coated.

The method of preparing a composite article may include providing a substrate including a first major surface and a second major surface, the substrate including a composition of 1-99 wt. % of an organic polymer and 1-99 wt. % of an inorganic filler. The method of preparing a composite article may further include applying an amorphous carbon coating over the first major surface or the second major surface to form a composite article.

The composite article may be modified or formed into a medical device or another device in a desired shape using any common techniques known in the art, including, but not limited to, cutting, forging, rolling, flanging, chamfering, turning, reaming, extruding, molding, sintering, or casting.

Use of the composite article and/or medical device according to embodiments herein may provide increased benefits in several applications in fields such as, but not limited to, industrial, medical, health care, biopharmaceutical, pharmaceutical, drinking water, food & beverage, laboratory, dairy, or other types of applications. According to embodiments herein, composite article and/or medical device are provided that may provide improved performance in chemical inertness, biocompatibility, elasticity, temperature resistance, and improve dissipation of static charge verses existing composite articles and/or medical devices. Further, the amorphous carbon coating may provide lower friction, higher hardness, and lower permeability versus existing composite articles and/or medical devices.

The above-disclosed subject matter is to be considered illustrative, and not restrictive, and the appended claims are intended to cover all such modifications, enhancements, and other embodiments, which fall within the true scope of the present invention. Thus, to the maximum extent allowed by law, the scope of the present invention is to be determined by the broadest permissible interpretation of the following claims and their equivalents, and shall not be restricted or limited by the foregoing detailed description.

In addition, in the foregoing Detailed Description, various features can be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter can be directed to less than all features of any of the disclosed embodiments. Thus, the following claims are incorporated into the Detailed Description, with each claim standing on its own as defining separately claimed subject matter.

Many different aspects and embodiments are possible. Some of those aspects and embodiments are described below. After reading this specification, skilled artisans will appreciate that those aspects and embodiments are only illustrative and do not limit the scope of the present invention. Embodiments may be in accordance with any one or more of the embodiments as listed below.

Embodiment 1. A composite article comprising:

a substrate, having a first major surface, a second major surface; and

an amorphous carbon coating overlying the first or the second major surface, wherein the substrate comprising a composition of 1-99 wt. % of an organic polymer and 1-99 wt. % of an inorganic filler.

Embodiment 2. A medical device comprising:

a composite article comprising a substrate, having a first major surface, a second major surface; and

an amorphous carbon coating overlying the first or the second major surface, wherein the substrate comprising a composition of 1-99 wt. % of an organic polymer and 1-99 wt. % of an inorganic filler.

Embodiment 3. The composite article or medical device according to any one of embodiments 1 or 2 wherein the organic polymer comprises a silicone elastomer.

Embodiment 4. The composite article or medical device according to embodiment 3, wherein the silicone elastomer comprises polydimethylsiloxane.

Embodiment 5. The composite article or medical device according to embodiment 3, wherein the inorganic filler comprises silica.

Embodiment 6. The composite article or medical device according to embodiment 3, wherein the silicone elastomer comprises hydride-containing polysiloxane cross-linker.

Embodiment 7. The composite article or medical device according to embodiment 3, wherein the silicone elastomer comprises a metal catalyst.

Embodiment 8. The composite article or medical device according to embodiment 3, wherein the silicone elastomer comprises a rubber.

Embodiment 9. The composite article or medical device according to embodiment 3, wherein a composite article has a Shore A hardness in the range from 5 to 95, in a range from 15 to 80, in a range from 25 to 70, or in a range from 40 to 60.

Embodiment 10. The composite article or medical device according to any one of the preceding embodiments, wherein the composite article has a coefficient of friction of 0.01 to 1.6 on a stainless steel surface.

Embodiment 11. The composite article or medical device according to any one of the preceding embodiments, wherein the composite article has a thickness of at least 0.025 mm, at least 0.050 mm, at least 0.100 mm, at least 0.500 mm, at least 1 mm, at least 2 mm, at least 3 mm, at least 4 mm, at least 5 mm, at least 6 mm, at least 8 mm, at least 10 mm, at least 50 mm, at least 80 mm, at least 100 mm, at least 150 mm, at least 200 mm, or at least 300 mm.

Embodiment 12. The composite article or medical device according to any one of the preceding embodiments, wherein the composite has a thickness of not greater than 1000 mm, not greater than 500 mm, not greater than 250 mm, not greater than 200 mm, or not greater than 1500 mm.

Embodiment 13. The composite article or medical device according to any one of the preceding embodiments, wherein the composite has a thickness in a range from 25 mm to 250 mm, in a range from 50 mm to 200 mm, in a range from 100 mm to 150 mm, or in a range from 100 mm to 125 mm.

Embodiment 14. The composite article or medical device according to any one of the preceding embodiments, wherein the substrate has a thickness of at least 25 mm, at least 30 mm, at least 35 mm, at least 40 mm, at least 45 mm, or at least 50 mm.

Embodiment 15. The composite article or medical device according to any one of the preceding embodiments, wherein the substrate has a thickness of not greater than 200 mm, not greater than 180 mm, not greater than 160 mm, not greater than 140 mm, not greater than 120 mm, not greater than 100 mm, not greater than 90 mm, not greater than 80 mm, not greater than 70 mm, or not greater than 60 mm.

Embodiment 16. The composite article or medical device according to any one of the preceding embodiments, wherein substrate is in a range from 20 mm to 200 mm, in a range from 25 mm to 180 mm, in a range from 30 mm to 120 mm, or in a range from 40 mm to 80 mm.

Embodiment 17. The composite article or medical device according to any one of the preceding embodiments, wherein the diamond-like carbon coating has a thickness of at least 10 Å, at least 20 Å, at least 30 Å, at least 50 Å, at least 80 Å, at least 100 Å, at least 150 Å, at least 200 Å, at least 300 Å, at least 400 Å, at least 500 Å, at least 600 Å, at least 800 Å, at least 1000 Å, at least 1200 Å, at least 1400 Å, at least 1600 Å, at least 1800 Å, or at least 2000 Å.

Embodiment 18. The composite article or medical device according to any one of the preceding embodiments, wherein the diamond-like carbon coating has a thickness of not greater than 5000 Å, not greater than 2500 Å, not greater than 2000 Å, not greater than 1800 Å, not greater than 1600 Å, not greater than 1400 Å, not greater than 1200 Å, not greater than 1000 Å, not greater than 800 Å, not greater than 700 Å, not greater than 650 Å, not greater than 600 Å, not greater than 550 Å, not greater than 500 Å, not greater than 480 Å, not greater than 460 Å, not greater than 440 Å, or not greater than 420 Å.

Embodiment 19. The composite article or medical device according to any one of the preceding embodiments, wherein the diamond-like carbon coating has a thickness in a range from 10 Å to 2000 Å, in a range from 50 Å to 1000 Å, in a range from 100 Å to 500 Å, or in a range from 100 Å to 200 Å.

Embodiment 20. The composite article or medical device according to any one of the preceding embodiments, wherein the substrate contains an organic polymer selected from an polydimethylsiloxane (PDMS), functionally modified polydimethylsiloxane, an polytetrafluoroethylene (PTFE), fluorinated ethylene-propylene (FEP), polyvinylidenfluoride (PVDF), polychlorotrifluoroethylene (PCTFE), ethylene chlorotrifluoroethylene (ECTFE), perfluoroalkoxypolymer (PFA), polyacetal (POM), polybutylene terephthalate (PBT), polyimide (PI), polyamidimide (PAI), polyetherimide, polyetheretherketone (PEEK), polyethylene, such as ultrahigh molecular weight polyethylene (UHMWPE), polysulfone, polyamide, polyphenylene oxide, polyphenylene sulfide (PPS), polyurethane, polyester, ethylene-tetrafluoroethylene (ETFE), tetrafluoro-ethylene-perfluoro (methyl vinyl ether) (MFA), polyvinylidene fluoride (PVDF), ethylene-chlorotrifluoroethylene (ECTFE), polyimide (PI), polyamidimide (PAI), polyphenylene sulfide (PPS), polyethersulofone (PES), polyphenylene sulfone (PPSO2), liquid crystal polymers (LCP), polyetherketone (PEK), polyether ether ketones (PEEK), aromatic polyesters (Ekonol), polyetherketone (PEK), liquid crystal polymer (LCP), polyamide (PA), polyethylene (PE), polypropylene (PP), polystyrene, styrene butadiene copolymers, polyesters, polycarbonate, polyacrylonitriles, polyamides, styrenic block copolymers, ethylene vinyl alcohol copolymers, ethylene vinyl acetate copolymers, polyesters grafted with maleic anhydride, poly-vinylidene chloride, aliphatic polyketone, liquid crystalline polymers, ethylene methyl acrylate copolymer, ethylene-norbornene copolymers, polymethylpentene and ethylene acrylic acid copoloymer, mixtures, copolymers, or any combination thereof.

Embodiment 21. The composite article or medical device according to embodiment 20, wherein the substrate contains a filler selected from glass, glass fibers, carbon, carbon fibers, silicon, graphite, PEEK, molybdenum disulfide, aromatic polyester, carbon particles, bronze, fluoropolymer, thermoplastic fillers, silicon carbide, aluminum oxide, polyamidimide (PAI), polyimide (PI), PPS, polyphenylene sulfone (PPSO2), liquid crystal polymers (LCP), aromatic polyesters (Econol), and mineral particles such as wollastonite, CaF₂, barium sulfate, or any combination thereof.

Embodiment 22. The composite article or medical device according to any one of the preceding embodiments, wherein the amorphous carbon coating has a position of the G band between 1505 cm⁻¹ and 1600 cm⁻¹ as measured by Raman spectroscopy.

Embodiment 23. The composite article or medical device according to any one of the preceding embodiments, wherein the amorphous carbon coating has a ratio of I (D)/I (G) by Raman spectroscopy ranging from 2.00 to 0.05 (I refers to the peak intensity).

Embodiment 24. The composite article or medical device according to any one of the preceding embodiments, wherein the composite article has substantial light transparency (60-80% light transmission in the visible range (TL)).

Embodiment 25. The composite article or medical device according to any one of the preceding embodiments, wherein the amorphous carbon coating has a refractive index between 1.2 and 2.5.

Embodiment 26. The composite article or medical device according to any one of the preceding embodiments, wherein the amorphous carbon coating has a density between 2.0 and 3.3 g/cm³.

Embodiment 27. The composite article or medical device according to any one of the preceding embodiments, wherein amorphous carbon coating generates higher surface resistivity compared to the substrate.

Embodiment 28. The composite article or medical device according to any one of the preceding embodiments, wherein the amorphous carbon coating has a carbon content of SP3 content of at least 2%, at least 5%, at least 10%, at least 20 wt. %, at least 30 wt. %, at least 40 wt. %, at least 50 wt. %, at least 60 wt. %, at least 70 wt. %, at least 75 wt. %, at least 80 wt. %, at least 85 wt. %, or at least 90 wt. %.

Embodiment 29. The composite article or medical device according to any one of the preceding embodiments, wherein the substrate has content of inorganic filler of at least 1 wt. %, 3 wt. %, 5 wt. %, 10 wt. %, 20 wt. %, at least 30 wt. %, at least 40 wt. %, at least 50 wt. %, at least 60 wt. %, at least 70 wt. %, at least 75 wt. %, at least 80 wt. %, at least 85 wt. %, or at least 90 wt. %, or at least 95 wt. %.

Embodiment 30. The composite article or medical device according to any one of the preceding embodiments, wherein the substrate has content of organic polymer of at least 5 wt. %, at least 10 wt. %, at least 20 wt. %, at least 30 wt. %, at least 40 wt. %, at least 50 wt. %, at least 60 wt. %, at least 70 wt. %, at least 75 wt. %, at least 80 wt. %, at least 85 wt. %, or at least 90 wt. %, or at least 95 wt. %, at least 99 wt. %.

Embodiment 31. The composite article or medical device according to any one of the preceding embodiments further comprising an adhesion promoter layer adjacent to the amorphous carbon coating.

Embodiment 32. The composite article or medical device according to embodiment 30, wherein the adhesion promoter layer comprises a thermoplastic.

Embodiment 33. The composite article or medical device according to embodiment 32, wherein the thermoplastic comprises tetrafluoroethylene-hexafluoropropylene (FEP), perfluoroalkoxyethylene (PFA), polyimide (PI), polyamidimide (PAI), polyphenylene sulfide (PPS), polyphenylene sulfone (PPSO2), polyether ether ketones (PEEK), aromatic polyesters (Ekonol), ethylene-tetrafluoroethylene (ETFE), tetrafluoro-ethylene-perfluoro (methyl vinyl ether) (MFA), modified polytetrafluoroethylene (TFM), polyvinylidene fluoride (PVDF), ethylene-chlorotrifluoroethylene (ECTFE), polyethersulfone (PES), polyetherketone (PEK), polyethylene (PE), UHMWPE, or any combination thereof.

Embodiment 34. The composite article or medical device according to embodiment 32, wherein the adhesion promoter layer comprises a metal element containing compound such as aluminum oxide, silicon oxide, or silanes, siloxane titanium oxide.

Embodiment 35. The composite article or medical device according to embodiment 32, wherein the thermoplastic includes a modified thermoplastic comprising at least one of the group selected from C(═O)R, C—O—R, COOH, COOR, COH, or any combination thereof, wherein R are cyclic or linear organic residues having from 1 to 20 carbon atoms.

Embodiment 36. The composite article or medical device according to any one of the preceding embodiments wherein the substrate comprises a discontinuous layer.

Embodiment 37. The composite article or medical device according to embodiment 36, wherein the discontinuous layer is selected from a mesh, a fleece, a foam, or any combination thereof.

Embodiment 38. The composite article or medical device according to embodiment 36, wherein the discontinuous layer has a mesh size of at least 10 mesh/inch, such as at least 11 mesh/inch, at least 13 mesh/inch, at least 15 mesh/inch, at least 17 mesh/inch, at least 19 mesh/inch, or at least 21 mesh/inch.

Embodiment 39. The composite article or medical device according to any one of embodiments 36-38, wherein the discontinuous layer has a structured surface.

Embodiment 40. The composite article or medical device according to any one of embodiments 36-39, wherein the discontinuous layer has a structured surface comprising wall-like depressions, wall-like elevations, or any combination thereof.

Embodiment 41. The composite article or medical device according to embodiment 40, wherein the wall-like depressions or wall-like elevations have a height in a range of 0.1 mm to 200 mm, in a range of 1 micron to 50 mm, or 2 mm to 30 mm.

Embodiment 42. The composite article or medical device according to any one of the preceding embodiments, wherein the discontinuous layer has a polygon structure.

Embodiment 43. The composite article or medical device according to embodiment 42, wherein the polygon structure is regular or non-regular.

Embodiment 44. The composite article or medical device according to embodiment 42, wherein the polygon structure is a honeycomb structure.

Embodiment 45. The composite article or medical device according to any one of the preceding embodiments, wherein the substrate comprises an edge between the first major surface and the second major surface, and wherein the amorphous carbon coating overlies the edges.

Embodiment 46. The composite article or medical device according to any one of the preceding embodiments, wherein the composite article surface electrical resistivity (Ω/Sq) between 10² and 10¹⁸.

Embodiment 47. The composite article or medical device according to any one of the preceding embodiments, wherein the composite article has a high temperature resistance between 150° C. and 260° C.

Embodiment 48. The composite article or medical device according to any one of the preceding embodiments, wherein the composite article has a low temperature resistance between −110° C. and −30° C.

Embodiment 49. The composite article or medical device according to any one of embodiments 2-48, wherein the composite article or medical device has a polygonal cross-section down a central axis.

Embodiment 50. The composite article or medical device according to embodiment 49, wherein the polygonal cross-section is regular or non-regular.

Embodiment 51. The composite article or medical device according to any one of embodiment 2-48, wherein the composite article or medical device has a circular or oval cross-section down a central axis.

Embodiment 52. The composite article or medical device according to any one of embodiments 2-48, wherein the composite article or medical device has an inner diameter, ID, wherein the inner diameter is at least 0.1 mm, at least 1 mm, at least 2 mm, at least 5 mm, at least 10 mm, at least 25 mm, at least 50 mm, at least 100 mm, at least 200 mm, at least 250 mm, or at least 500 mm.

Embodiment 53. The composite article or medical device according to embodiment 52, wherein the composite article or medical device has an inner diameter, ID, wherein the inner diameter of not greater than 1000 mm, not greater than 500 mm, not greater than 250 mm, not greater than 200 mm, or not greater than 1500 mm.

Embodiment 54. The composite article or medical device according to embodiment 52, wherein the composite article or medical device has an inner diameter, ID, wherein the inner diameter is in a range from 0.1 mm to 250 mm, in a range from 1 mm to 200 mm, in a range from 1 mm to 150 mm, or in a range from 1 mm to 125 mm.

Embodiment 55. The composite article or medical device according to embodiment 52, wherein the composite article or medical device has an inner diameter, ID, that varies along a length of the medical device.

Embodiment 56. The composite article or medical device according to according to any one of embodiments 2-50, wherein the composite article or medical device has an outer diameter, OD, wherein the outer diameter is at least 0.5 mm, at least 1 mm, at least 2 mm, at least 5 mm, at least 10 mm, at least 25 mm, at least 50 mm, at least 100 mm, at least 200 mm, at least 250 mm, or at least 500 mm.

Embodiment 57. The composite article or medical device according to embodiment 56, wherein the composite article or medical device has an outer diameter, OD, wherein the outer diameter of not greater than 1000 mm, not greater than 500 mm, not greater than 250 mm, not greater than 200 mm, or not greater than 1500 mm.

Embodiment 58. The composite article or medical device according to embodiment 56, wherein the composite article or medical device has an outer diameter, OD, wherein the outer diameter is in a range from 1 mm to 250 mm, in a range from 1 mm to 200 mm, in a range from 5 mm to 150 mm, or in a range from 5 mm to 125 mm.

Embodiment 59. The composite article or medical device according to embodiment 56, wherein the composite article or medical device has an outer diameter, OD, that varies along a length of the medical device.

Embodiment 60. The medical device according to any one of the previous embodiments, wherein the medical device comprises an infusion sleeve.

Embodiment 61. The medical device according to any one of the previous embodiments, wherein the medical device comprises structures having a lumen through which a fluid may be carried, pumped, or otherwise transported through.

Embodiment 62. The medical device according to any one of the previous embodiments, wherein the medical device has substantial transparency.

Embodiment 63. The medical device according to any one of the previous embodiments, wherein the medical device is sterile.

Embodiment 64. A method of preparing a composite article, the method comprising:

providing a substrate comprising a first major surface and a second major surface, the substrate comprising a composition of 1-99 wt. % of an organic polymer and 1-99 wt. % of an inorganic filler;

applying an amorphous carbon coating over the first major surface or the second major surface to form a composite article.

Embodiment 65. The method according to embodiment 64, wherein the coating includes a surface treatment including physical vapor deposition, chemical vapor disposition, plasma enhanced chemical vapor disposition, spraying, microwave radiation, ultraviolet radiation, x-ray radiation, gamma radiation, alpha radiation, beta radiation, charged ions, neutron radiation, vacuum plasma radiation, other chemical or electrochemical techniques, or any combination thereof.

Embodiment 66. The method according to embodiment 64, further comprising applying an amorphous carbon coating over an edge of the substrate located between the first major surface and the second major surface.

Embodiment 67. A medical tube comprising:

a substrate comprising a first major surface and a second major surface, the substrate comprising a composition of 1-99 wt. % of an organic polymer and 1-99 wt. % of an inorganic filler; and

an amorphous carbon coating over the first major surface or the second major surface to form a composite article.

Embodiment 68. The medical tube as described in embodiment 67, characterized in that the composite article has at least one axial collar.

Embodiment 69. The medical tube as described in embodiment 67 or 68, characterized in that the composite article has an essentially cylindrical shape.

Embodiment 70. The medical tube as described in embodiment 67 or 68, characterized in that the composite article has a conical shape.

Embodiment 71. An infusion sleeve comprising:

a substrate comprising a first major surface and a second major surface, the substrate comprising a composition of 1-99 wt. % of an organic polymer and 1-99 wt. % of an inorganic filler; and

an amorphous carbon coating over the first major surface or the second major surface to form a composite article.

Embodiment 72. The infusion sleeve as described in embodiment 71, characterized in that the composite article has at least one axial collar.

Embodiment 73. The infusion sleeve as described in embodiment 71 or 72, characterized in that the composite article has an essentially cylindrical shape.

Embodiment 74. The infusion sleeve as described in embodiment 71 or 72, characterized in that the composite article has a conical shape.

Note that not all of the features described above are required, that a portion of a specific feature may not be required, and that one or more features may be provided in addition to those described. Still further, the order in which features are described is not necessarily the order in which the features are disclosed.

Certain features are, for clarity, described herein in the context of separate embodiments, may also be provided in combination in a single embodiment. Conversely, various features that are, for brevity, described in the context of a single embodiment, may also be provided separately or in any subcombinations.

Benefits, other advantages, and solutions to problems have been described above with regard to specific embodiments, However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range, including the end range values referenced. Many other embodiments may be apparent to skilled artisans only after reading this specification. Other embodiments may be used and derived from the disclosure, such that a structural substitution, logical substitution, or any change may be made without departing from the scope of the disclosure. Accordingly, the disclosure is to be regarded as illustrative rather than restrictive.

What is claimed is:

1. A composite article comprising:
a substrate, having a first major surface, a second major surface; and an amorphous carbon coating overlying the first or the second major surface,
wherein the substrate comprising a composition of 1-99 wt. % of an organic polymer and 1-99 wt. % of an inorganic filler.

2. A medical device comprising:
a composite article comprising a substrate, having a first major surface, a second major surface; and
an amorphous carbon coating overlying the first or the second major surface, wherein the substrate comprising a composition of 1-99 wt. % of an organic polymer and 1-99 wt. % of an inorganic filler.

3. The composite article according to claim 1, wherein the organic polymer comprises a silicone elastomer.

4. The composite article according to claim 3, wherein the silicone elastomer comprises polydimethylsiloxane.

5. The composite article according to claim 3, wherein the inorganic filler comprises silica.

6. The composite article according to claim 3, wherein the silicone elastomer comprises hydride-containing polysiloxane cross-linker.

7. The composite article according to claim 3, wherein the silicone elastomer comprises a metal catalyst.

8. The composite article according to claim 3, wherein the silicone elastomer comprises a rubber.

9. The composite article according to claim 3, wherein a composite article has a Shore A hardness in the range from 5 to 95.

10. The composite article according to claim 1, wherein the composite article has a coefficient of friction of 0.01 to 1.6 against stainless steel surface.

11. The composite article according to claim 1, wherein the composite article has a thickness of at least 0.025 mm.

12. The composite article according to claim 1, wherein the composite has a thickness of not greater than 1000 mm.

13. The composite article according to claim 1, where the amorphous carbon coating comprises hydrogen (a-C:H or hydrogenated amorphous carbon).

14. The composite article according to claim 1, wherein the amorphous carbon coating has a thickness of at least 10 Å.

15. The composite article according to claim 1, wherein the amorphous carbon coating has a thickness of not greater than 5000 Å.

16. The composite article according to claim 1, wherein the amorphous carbon coating has a thickness in a range from 10 Å to 2000 Å.

17. The composite article according to claim 1, wherein the amorphous carbon coating has a position of the G band between 1505 cm⁻¹ and 1600 cm⁻¹ as measured by Raman spectroscopy.

18. The composite article according to claim 1, wherein the amorphous carbon coating has a ratio of I (D)/I (G) by Raman spectroscopy ranging from 2.00 to 0.05.

19. The composite article according to claim 1, wherein the composite article has substantial light transparency (60-80% light transmission in the visible range (TL)).

20. The composite article according to claim 1, wherein the substrate contains an organic polymer selected from an polydimethylsiloxane (PDMS), functionally modified polydimethylsiloxane, an polytetrafluoroethylene (PTFE), fluorinated ethylene-propylene (FEP), polyvinylidenfluoride (PVDF), polychlorotrifluoroethylene (PCTFE), ethylene chlorotrifluoroethylene (ECTFE), perfluoroalkoxypolymer (PFA), polyacetal (POM), polybutylene terephthalate (PBT), polyimide (PI), polyamidimide (PAI), polyetherimide, polyetheretherketone (PEEK), polyethylene, such as ultrahigh molecular weight polyethylene (UHMWPE), polysulfone, polyamide, polyphenylene oxide, polyphenylene sulfide (PPS), polyurethane, polyester, ethylene-tetrafluoroethylene (ETFE), tetrafluoro-ethylene-perfluoro (methyl vinyl ether) (MFA), polyvinylidene fluoride (PVDF), ethylene-chlorotrifluoroethylene (ECTFE), polyimide (PI), polyamidimide (PAI), polyphenylene sulfide (PPS), polyethersulofone (PES), polyphenylene sulfone (PPSO2), liquid crystal polymers (LCP), polyetherketone (PEK), polyether ether ketones (PEEK), aromatic polyesters (Ekonol), polyetherketone (PEK), liquid crystal polymer (LCP), polyamide (PA), polyethylene (PE), polypropylene (PP), polystyrene, styrene butadiene copolymers, polyesters, polycarbonate, polyacrylonitriles, polyamides, styrenic block copolymers, ethylene vinyl alcohol copolymers, ethylene vinyl acetate copolymers, polyesters grafted with maleic anhydride, poly-vinylidene chloride, aliphatic polyketone, liquid crystalline polymers, ethylene methyl acrylate copolymer, ethylene-norbornene copolymers, polymethylpentene and ethylene acrylic acid copoloymer, mixtures, copolymers, or any combination thereof.
